(19) United States
(12) Patent Application Publication
(10) Pub. No.: US 2024/0331811 A1
(43) Pub. Date: Oct. 3, 2024

(54) METHOD AND SERVER FOR GENERATING, ON BASIS OF LANGUAGE MODEL, QUESTIONS OF PERSONALITY APTITUDE TEST BY USING QUESTION AND ANSWER NETWORK

(71) Applicant: GLORANG INC., Seoul (KR)

(72) Inventors: Yong-hoon KWON, Seoul (KR); Sung-uk CHOI, Seoul (KR); Dong-jin SEO, Seoul (KR); Tae-il HWANG, Seoul (KR)

(21) Appl. No.: 18/681,176

(22) PCT Filed: Sep. 5, 2023
(86) PCT No.: PCT/KR2023/013241

§ 371 Date: Feb. 5, 2024

(30) Foreign Application Priority Data
Sep. 14, 2022 (KR) 10-2022-0115444
Jul. 10, 2023 (KR) 10-2023-0088846

(51) Int. Cl. G16H 10/20

(52) U.S. Cl. CPC G16H 10/20

(57) ABSTRACT

A method and server for generating a question for personality and aptitude tests using a question and answer network based on a language model are disclosed. The server according to the present disclosure includes a communication unit configured to communicate with a terminal, a database configured to store tester information, a memory configured to store artificial intelligence model data for a generative artificial intelligence model, and a processor configured to generate personality and aptitude question information suitable for characteristics of a person from personal behavior characteristic information and existing question information by using the generative artificial intelligence model.

### TECHNICAL FIELD

The present disclosure relates to an electronic device and an operating method thereof. More particularly, the present disclosure relates to a method and server for generating a question for personality and aptitude tests using a question and answer network based on a language model.

### BACKGROUND ART

If a personality and aptitude question is developed, it is important to define lower factors by using the existing reference question. To this end, characteristics of a target to be tested need to be checked, and proper reference needs to be selected. Thereafter, a method of developing a personality and aptitude question includes extracting lower factors by analyzing a selected reference question and deriving an expected behavior characteristic of a tester based on the lower factors.

A proper question is generated based on the extracted lower factors and the expected behavior characteristics. To this end, it is necessary to consider a construction, the type, a difficulty, objectivity, validity, etc. of the question. Recently, as the number of parameters of a language model is increased, large language models (LLMs) show a capability for learning in context through some examples. The corresponding capability is called “in context learning”. In a corresponding method, a result is predicted by inputting some examples to the LLMs that have been previously trained. The corresponding method has an advantage in that the method can be applied to real services because the update of a parameter does not occur and a calculation cost can be reduced unlike the existing supervised learning.

However, the corresponding method may have a problem with many costs because psychologists directly perform processes from a process of setting lower factors of a question up to a process of deriving behavior characteristics based on the factors in a process of generating a new question.

### DISCLOSURE

### Technical Problem

An object of the present disclosure is to provide a method and server for solving a cost problem through artificial intelligence and developing a personality and aptitude question that is suitable for a person by additionally using behavior information of the person rather than suggesting a common question.

### Technical Solution

In an aspect, a server of the present disclosure includes a communication unit configured to communicate with a terminal, a database configured to store tester information, a memory configured to store artificial intelligence model data for a generative artificial intelligence model, and a processor configured to generate personality and aptitude question information suitable for characteristics of a person from personal behavior characteristic information and existing question information by using the generative artificial intelligence model. The processor receives tester question information and a request for a personality and aptitude question for personality and aptitude tests from the terminal, inputs, to the generative artificial intelligence model, the tester information corresponding to the terminal, which are extracted from the database, and the tester question information, and transmits, to the terminal, the personality and aptitude question information suitable for the characteristics of a tester who uses the terminal, which is provided as an output of the generative artificial intelligence model, through the communication unit.

In another aspect, a method of generating personality and aptitude question information suitable for characteristics of a person from personal behavior characteristic information and existing question information by using a generative artificial intelligence model of the present disclosure includes receiving tester question information and a request for a personality and aptitude question for personality and aptitude tests from a terminal, inputting, to the generative artificial intelligence model, tester information corresponding to the terminal, which are extracted from a database, and the tester question information, and generates personality and aptitude question information suitable for characteristics of a tester who uses the terminal, which is provided as an output of the generative artificial intelligence model.

### Advantageous Effects

According to the present disclosure, there are effects in that a question can be generated at a lower cost than a cost when the existing professional manpower is input because a personality and aptitude question is generated by using artificial intelligence and a customized test for each user can be performed because of the question in which the characteristics of a person have been incorporated.

### DESCRIPTION OF DRAWINGS

FIG. 1 is a diagram illustrating a system according to the present disclosure.

FIG. 2 illustrates a construction of a server of FIG. 1.

FIG. 3 is a flowchart illustrating a method according to the present disclosure.

FIGS. 4 and 5 are diagrams illustrating examples of a process of examining, by an expert, a corresponding question for personality and aptitude tests, which has been learnt and recommended through an artificial intelligence-based question development model of a processor of FIG. 2.

FIGS. 6 and 7 are diagrams illustrating examples of a process of displaying a corresponding question for personality and aptitude tests on a terminal of a user so that the user solves the corresponding question for personality and aptitude tests which has been extracted from the server of FIG. 2.

FIG. 8 is a block diagram for describing the system of the present disclosure.

FIG. 9 is a diagram for describing an embodiment of the present disclosure in which all questions are provided.

FIG. 10 is a diagram for describing an embodiment of the present disclosure in which a loop type question is provided.

FIG. 11 is a diagram for describing a model architecture having a Decode-Only structure according to the present disclosure.

FIGS. 12 and 13 are diagrams for describing embodiments in which a question is generated by using a generative artificial intelligence model of the present disclosure.

FIG. 14 is a diagram for describing input values for input embedding and behavioral embedding according to the present disclosure.

FIGS. 15, 16, 17, and 18 are diagrams for illustratively describing end conditions according to the present disclosure.

### BEST MODE FOR INVENTION

In this specification, “an apparatus according to the present disclosure” includes all of various apparatuses which can provide results to a user by performing operation processing. For example, the apparatus according to the present disclosure may include all of a computer, a server apparatus, and a mobile terminal or may have any one form of a computer, a server apparatus, and a mobile terminal.

A function related to artificial intelligence according to the present disclosure is operated through a processor and memory. The processor may include one or a plurality of processors. In this case, the one or the plurality of processors may be general-purpose processors, such as a CPU, an AP, and a DSP, graphic-dedicated processors, such as a GPU and a VPU, and artificial intelligence-dedicated processors, such as an NPU. The one or the plurality of processors controls input data to be processed according to a predefined operation rule or artificial intelligence model that has been stored in the memory. Alternatively, if the one or the plurality of processors is artificial intelligence-dedicated processors, the artificial intelligence-dedicated processors may be designed to have a hardware structure that has been specialized for the processing of a specific artificial intelligence model.

According to an exemplary embodiment of the present disclosure, the processor may implement artificial intelligence. Artificial intelligence means an artificial neural network-based machine learning method of imitating a neuron of a person so that a machine can be trained. A methodology for artificial intelligence may be divided into supervised learning, unsupervised learning, and reinforcement learning depending on a learning method. Furthermore, the methodology for artificial intelligence may be divided based on architecture, that is, a structure of a learning model. Architecture for a deep learning technology, which is widely used, may be divided into a convolutional neural network (CNN), a recurrent neural network (RNN), a transformer, a generative alternative neural network (GAN), etc.

The present apparatus and system may include an artificial intelligence model. The artificial intelligence model may be one artificial intelligence model, and may be implemented as a plurality of artificial intelligence models. The artificial intelligence model may consist of a neural network (or an artificial neural network), and may include a statistical learning algorithm that imitates a nerve of biology in the machine learning and cognitive science. The neural network may mean all models in each of which artificial neurons (nodes) that have formed a network through a combination of synapses have a question-solving ability by changing the coupling intensity of the synapses. The neuron of the neural network may include a combination of weights or biases. The neural network may include one or more layers each consisting of one or more neurons or nodes. Illustratively, the apparatus may include an input layer, a hidden layer, and an output layer. The neural network that constitutes the apparatus may infer a result (output) to be predicted from an arbitrary input by changing the weight of a neuron through learning.

The processor may generate a neural network, train (or learn) the neural network, or perform an operation based on received input data, may generate an information signal based on the results of the execution, or may retrain the neural network. Models of the neural network may include various types of models, such as a convolution neural network (CNN), a region with convolution neural network (R-CNN), a region proposal network (RPN), a recurrent neural network (RNN), a stacking-based deep neural network (S-DNN), a state-space dynamic neural network (S-SDNN), a deconvolution network, a deep belief network (DBN), a restricted Boltzman machine (RBM), a fully convolutional network, a long short-term memory (LSTM) Network, and a classification network, such as GoogleNet, AlexNet, and VGG Network, but the present disclosure is not limited thereto. The processor may include one or more processors for performing an operation according to models of a neural network. For example, the neural network may include a deep neural network.

According to an exemplary embodiment of the present disclosure, the processor may use various artificial intelligence structures and algorithms, such as a CNN, an R-CNN, an RPN, an RNN, an S-DNN, an S-SDNN, a deconvolution network, a DBN, RBM, a fully convolutional network, an LSTM network, a classification network, Generative Modeling, eXplainable AI, continual AI, representation learning, and AI for material design, such as GoogleNet, AlexNet, and a VGG Network, BERT, SP-BERT, MRC/QA, Text Analysis, Dialog System, GPT-3, and GPT-4 for natural language processing, Visual Analytics, Visual Understanding, and Video Synthesis vision processing, and Anomaly Detection, Prediction, Time-Series Forecasting, Optimization, Recommendation, Data Creation for ResNet data intelligence, and the present disclosure is not limited thereto. Embodiments of the present disclosure are described in detail with reference to the drawings.

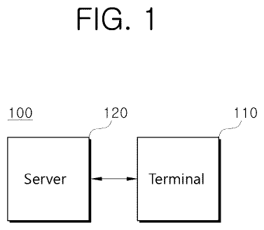

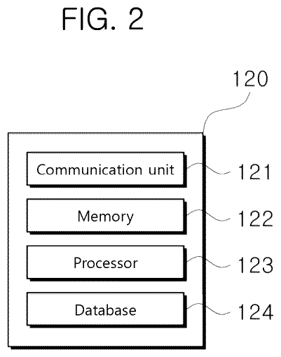

FIG. 1 is a diagram illustrating a system according to the present disclosure. FIG. 2 illustrates a construction of a server of FIG. 1.

Referring to FIGS. 1 and 2, a new development system 100 for a personality and aptitude question based on artificial intelligence may include a terminal 110 of a user and a server 120.

The terminal 110 of a user may request the suggestion of a question for personality and aptitude tests for the personality and aptitude tests. The terminal 110 may transmit tester question information and a personality and aptitude question request for the personality and aptitude tests to the server 120. The terminal 110 is a wireless communication apparatus having guaranteed portability and mobility, and may include all types of handheld-based wireless communication apparatuses, such as a personal communication system (PCS), global system for mobile communications (GSM), personal digital cellular (PDC), and a smartphone, and wearable apparatuses, such as a watch and a ring.

The server 120 may include a communication unit 121, memory 122, a processor 123, and a database 124.

The communication unit 121 may perform communication with the terminal 110. In this case, the communication unit 121 may include wireless communication modules that support various wireless communication methods, such as global system for mobile communications (GSM), code division multiple access (CDMA), wideband code division multiple access (WCDMA), a universal mobile telecommunications system (UMTS), time division multiple access (TDMA), long term evolution (LTE), 4G, 5G, and 6G, in addition to a Wi-Fi module and a wireless broadband module.

The memory 122 may store data of an algorithm for controlling an operation of components within the present apparatus or data of a program that has reproduced the algorithm, and may be implemented by at least one processor 123 that performs the aforementioned operation by using data stored in the memory 122. In this case, the memory 122 and the processor 123 may be implemented as separate chips. Furthermore, the memory 122 and the processor 123 may be implemented as a single chip.

The memory 122 may store data that support various functions of the present apparatus and a program for an operation of the processor 123, may store data that are input and output, and may store multiple application programs (or applications) that are driven in the present apparatus and data and instructions for an operation of the present apparatus. At least some of such application programs may be downloaded from an external server through wireless communication.

The memory 122 may include at least one type of storage medium, among a flash memory type, a hard disk type, a solid state disk (SSD) type, a silicon disk drive (SDD) type, a multimedia card micro type, card type memory (e.g., SD or XD memory), random access memory (RAM), static random access memory (SRAM), read-only memory (ROM), electrically erasable programmable read-only memory (EEPROM), programmable read-only memory (PROM), magnetic memory, a magnetic disk, and an optical disk. Furthermore, the memory 122 has been separated from the present apparatus, but may be a database that has been connected to the present apparatus in a wired or wireless way.

The memory 122 may store artificial intelligence model data for a generative artificial intelligence model.

The processor 123 may control an operation related to a process of new developing, testing, and feeding back a personality and aptitude question. The processor 123 may receive a request for the suggestion of a question for personality and aptitude tests for the personality and aptitude tests from the terminal 110 of a user, and may extract a corresponding question for personality and aptitude tests, which is learnt and recommended through an artificial intelligence-based question development model based on the propensity of the terminal 110 of a user. The processor 123 may display the corresponding question for personality and aptitude tests on the terminal 110 of a user so that the user can solve the corresponding question for personality and aptitude tests.

The processor 123 may be configured to generate personality and aptitude question information that is suitable for the characteristics of a person based on personal behavior characteristic information and the existing question information by using a generative artificial intelligence model. Specifically, for example, the processor 123 may receive tester question information and a request for a personality and aptitude question for personality and aptitude tests from the terminal 110. Furthermore, the processor 123 may input, to the generative artificial intelligence model, tester information corresponding to the terminal 110 in the database 124, and may input the tester question information to the generative artificial intelligence model. The generative artificial intelligence model may output personality and aptitude question information that is suitable for the characteristics of the tester who uses the terminal 110. That is, the personality and aptitude question information that is suitable for the characteristics of the tester who uses the terminal 110 may be generated as the output of the generative artificial intelligence model. The processor 123 may transmit, to the terminal 110, the personality and aptitude question information that is suitable for the characteristics of the tester who uses the terminal 110 through the communication unit 121.

The database 124 may be configured to store tester information. The tester information may include at least one of a course history, search history, and list of interests of a tester, for example. The tester information may be previously stored in the database 124.

At least one component may be added or deleted in accordance with performance of the components illustrated in FIGS. 1 and 2. Furthermore, a person having ordinary knowledge in the art may easily understand that mutual locations of the components may be changed in accordance with performance or a structure of the system.

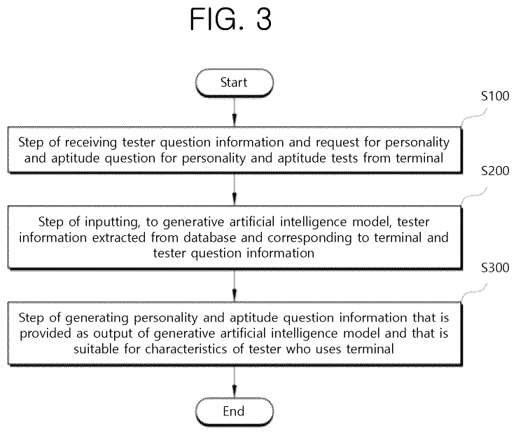

FIG. 3 is a flowchart illustrating a method according to the present disclosure.

Referring to FIG. 3, the method of FIG. 3 may be performed by the server 120 of FIGS. 1 and 2. The method of FIG. 3 may be a method of performing a sampling process for personality and aptitude tests using a question and answer network based on a language model. Furthermore, the method of FIG. 3 may be a method of generating personality and aptitude question information that is suitable for the characteristics of a person from personal behavior characteristic information and the existing question information by using a generative artificial intelligence model.

A step of receiving tester question information and a request for a personality and aptitude question for personality and aptitude tests from the terminal is performed (S100).

A step of inputting tester information that is extracted from the database and that corresponds to the terminal and the tester question information to the generative artificial intelligence model is performed (S200).

A step of generating personality and aptitude question information that is provided as the output of the generative artificial intelligence model and that is suitable for the characteristics of the tester who uses the terminal is performed (S300).

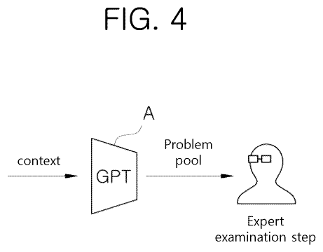

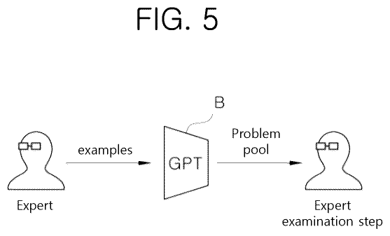

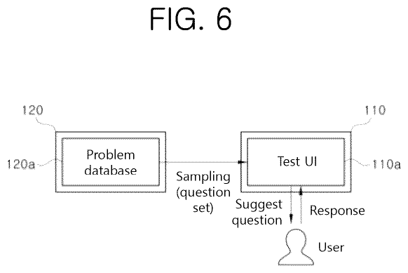

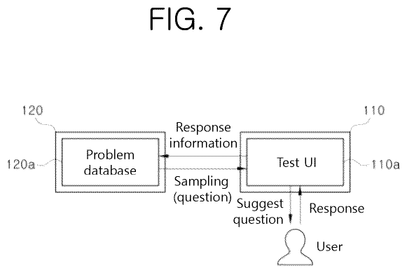

FIGS. 4 and 5 are diagrams illustrating examples of a process of examining, by an expert, a corresponding question for personality and aptitude tests, which has been learnt and recommended through an artificial intelligence-based question development model of the processor of FIG. 2. FIGS. 6 and 7 are diagrams illustrating examples of a process of displaying a corresponding question for personality and aptitude tests on the terminal of a user so that the user solves the corresponding question for personality and aptitude tests which has been extracted from the server of FIG. 2.

Referring to FIGS. 4 to 7, the processor 123 may receive a request for the suggestion of a question for personality and aptitude tests for the personality and aptitude tests from the terminal 110 of a user. The processor 123 may extract the corresponding question for personality and aptitude tests, which has been learnt and recommended through an artificial intelligence-based question development model based on the propensity of the terminal 110. In this case, in the process of extracting the question for personality and aptitude tests, the corresponding question for personality and aptitude tests, which has been learnt and recommended through at least one method of zero-shot learning, few-shot learning, and one-shot learning within the question development model, may be extracted. Furthermore, in the process of extracting the question for personality and aptitude tests, a question for personality and aptitude tests may be generated and extracted one by one in real time based on the propensity of the terminal 110 of a user whenever a request for the suggestion of the question for personality and aptitude tests is received. In this case, the artificial intelligence-based question development model may include a generative pre-trained transformer (GPT) model. In this case, the GPT model is a language model, and is pre-trained in a process of trying to figure out what a next word is when previous words are given. The GPT model has one-way property in that calculation is sequentially performed from the start of a sentence.

As illustrated in FIGS. 4 and 5, the GPT model A, B generates results in which an output for a prompt is very stable and repeatedly suitable if only some examples are given. Such an example-based conditioning form has an advantage in that it can be directly applied to several tasks. The GPT model A, B may generate a next question after checking that a user solves a question. Furthermore, the GPT model A, B may generate a list of questions differently for each user. In the present disclosure, an expert may examine a question for personality and aptitude tests, which has been learnt and recommended through the GPT model A, B.

As illustrated in FIG. 6, when receiving a request for the suggestion of a question for personality and aptitude tests from the terminal 110 of a user, the server 120 may extract the corresponding question for personality and aptitude tests from a question database 120a, which has been trained and databased through an artificial intelligence-based question development model, and may transmit the extracted corresponding question for personality and aptitude tests to the terminal 110 of the user. The terminal 110 of the user may display the corresponding question for personality and aptitude tests through a test UI 110a. The user (or a tester) may input a response to the corresponding question for personality and aptitude tests through the test UI 110a.

As illustrated in FIG. 7, the server 120 may include a generative model that suggests next questions one by one on the basis of a real-time response from a user. That is, the server 120 may present one question to the terminal 110 of a user by sampling the question as a unit on the basis of response information. In this case, in the present disclosure, a method of a neural network operating at the backend of the test UI 110a may be performed because a kind of determination for the user needs to be made.

The present disclosure can reduce a cost for developing a personality and aptitude question because the personality and aptitude question can be developed and examined easily and newly.

The processor 123 may implement the aforementioned artificial intelligence. As described above, methodologies for artificial intelligence may be divided into supervised learning, non-supervised learning, and reinforcement learning. Architectures for a deep learning technology may be divided into a CNN, an RNN, a transformer, a GAN, etc. An artificial intelligence model may be one or more artificial intelligence models.

As described above, the processor 123 may generate a neural network, train (or learn) the neural network, or perform an operation based on received input data, may generate an information signal based on the results of the execution, or may retrain the neural network. The neural network may include a CNN, an RNN, perceptron, multi-layer perceptron, etc., but the present disclosure is not limited thereto. Those skilled in the art will understand that the neural network may include an arbitrary neural network.

The processor 123 may use various artificial intelligence structures and algorithms, such as a CNN, an R-CNN, an RPN, an RNN, an S-DNN, an S-SDNN, a deconvolution network, a DBN, RBM, a fully convolutional network, an LSTM network, a classification network, Generative Modeling, eXplainable AI, continual AI, representation learning, and AI for material design, such as GoogleNet, AlexNet, and a VGG Network, BERT, SP-BERT, MRC/QA, Text Analysis, Dialog System, GPT-3, and GPT-4 for natural language processing, Visual Analytics, Visual Understanding, and Video Synthesis vision processing, and Anomaly Detection, Prediction, Time-Series Forecasting, Optimization, Recommendation, Data Creation for ResNet data intelligence as described above, but the present disclosure is not limited thereto.

The CNN may be formed to have a structure in which a pooling layer that extracts a feature invariable to a change in the location or rotation is alternately repeated several times by spatially integrating a convolution layer that produces a feature map by applying a plurality of filters to each region of an image and a feature map. Accordingly, the CNN may extract features having various levels from a feature having a low level, such as a dot, a line, or a surface, to a feature having a high level, which is complicated and meaningful.

The convolution layer may calculate a feature map by applying a non-linear activation function to a filter and an inner product of a local receptive field with respect to each patch of an input image.

Compared to another network structure, the CNN may have a feature that uses a filter having sparse connectivity and shared weights. Such a connection structure can reduce the number of parameters to be learnt, and can resultantly improve prediction performance by making learning through a back-propagation algorithm efficient.

The feature that has been finally extracted through the repetition of the convolution layer and the pooling layer as described above may be combined with a classification model, such as multi-layer perceptron (MLP) or a support vector machine (SVM), which has a form of a fully-connected layer, and may be used for the training and prediction of a compression model.

Meanwhile, the artificial intelligence-based question development model may mean an artificial intelligence model that has been trained based on deep learning, and may mean a model that has been trained by using a convolutional neural network (CNN), for example. Furthermore, the artificial intelligence-based question development model may include at least one algorithm, among natural language processing (NLP), random forest (RF), a support vector machine (SVC), extra gradient boost (XGB), a decision tree (DC), Knearest neighbors (KNN), Gaussian Naive Bayes (GNB), a stochastic gradient descent (SGD), Linear discriminant analysis (LDA), a ridge, Lasso, and an elastic net.

The processor 123 may feed the results of a corresponding question for personality and aptitude tests, which has been solved by a corresponding user, back with respect to the personality and aptitude tests.

Meanwhile, in the present disclosure, the artificial intelligence-based question development model may be designed by considering behavior characteristics.

That is, an impulsive user may show a behavior characteristic that the user tries to do something right away or having difficulty resisting the urge. If a factor of impulsiveness is to be measured as a subfactor of a psychological test, a question may be generated in a form in which the question describes such a behavior characteristic. For example, a question having a form, such as “1) If there is something you want to do, you should do it right away.!” “2) I can't stand anything I want to buy.”, may be generated.

Meanwhile, in the present disclosure, the artificial intelligence-based question development model may be designed based on an expert test process.

That is, statistics analysis may be most used for an expert test process. For example, the statistics analysis may include factor analysis, reliability analysis, and convergent validity analysis. The factor analysis may be a process of binding questions having high correlation and checking which questions are appropriate to represent each factor. The reliability analysis may be a process of checking how consistently what it is intended to measure is measured. The convergent validity analysis may be a process of checking whether what it is intended to measure is measured properly by analyzing a correlation between a factor to be measured in a developed test and a measured test score of the same characteristic.

Meanwhile, in the present disclosure, the artificial intelligence-based question development model may be designed based on an impulsive test or a capability test.

That is, the impulsive test (MFFT) is a tool test. In this test, a tester shows several pictures to a user so that the user selects the same picture as a picture above. A correct answer is one of six pictures. The type of impulsiveness may be classified depending on a reaction speed and the number of correct answers.

The capability test is to solve a problem including a correct answer and to receive corresponding test results in an intelligence test, an ability test, etc. The capability test is different from a self-report type test having a response of 1 to 4 points.

Meanwhile, in the present disclosure, the artificial intelligence-based question development model may be designed by considering the severity of a special user.

That is, the test results of a special user are indicated as a standard score that is calculated based on an average or standard deviation (criterion) of a group of common peer users, and may be calculated to have a higher or lower score when a score of a psychological test factor deviates from an average by a 1 standard deviation.

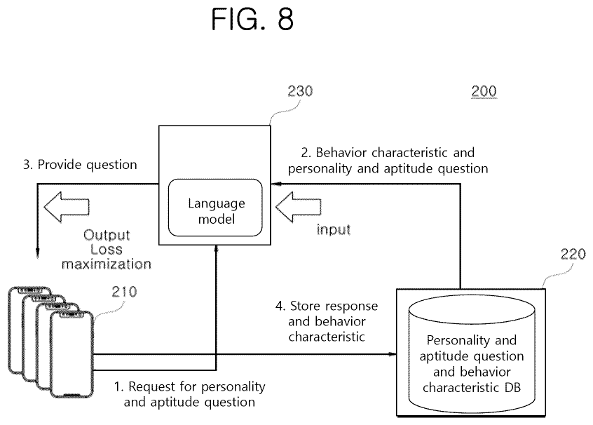

FIG. 8 is a block diagram for describing a system of the present disclosure.

Referring to FIG. 8, in a system 200, a terminal 210 may be an apparatus that is used by a tester. The tester may request a personality and aptitude question for each person by using the terminal 210.

An apparatus 230 may include a language model. The language model may receive behavior characteristics and a personality and aptitude question as an input from a personality and aptitude question and behavior characteristic database 220. The apparatus 230 may provide the personality and aptitude question for each person to the terminal 210 as an output. The output of the language model may be based on loss maximization.

The tester may respond to the personality and aptitude question for each person, which has been received from the apparatus 230, through the terminal 210. Furthermore, the tester may store the response and the behavior characteristic in the database 220 through the terminal 210.

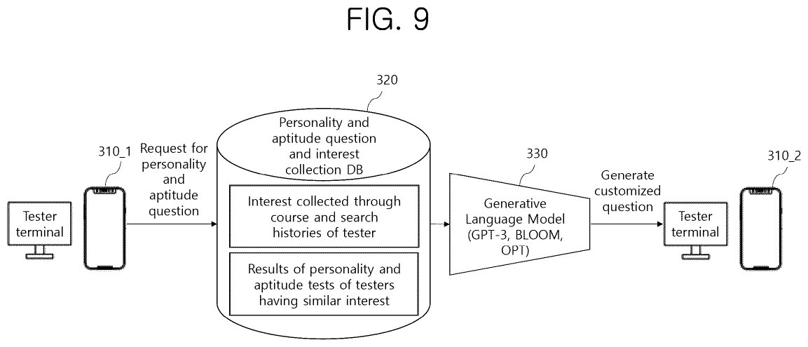

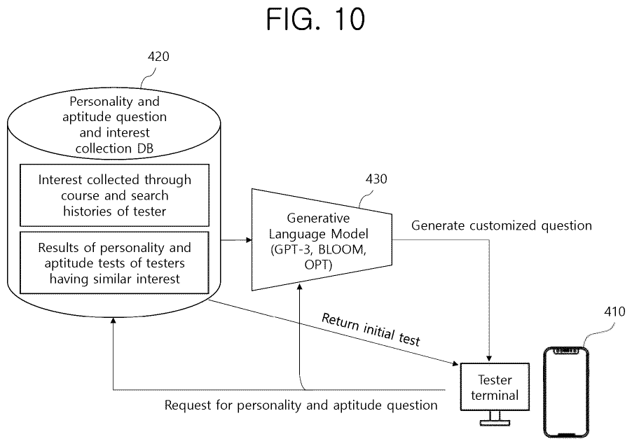

FIG. 9 is a diagram for describing an embodiment of the present disclosure in which all questions are provided. FIG. 10 is a diagram for describing an embodiment of the present disclosure in which a loop type question is provided.

Referring to FIG. 9, a tester terminal 310_1 may provide a request for a personality and aptitude question to a personality and aptitude question and interest collection database 320. The personality and aptitude question and interest collection database 320 may store interest that is collected through courses and search histories of the tester and information on the personality and aptitude tests results of testers who have similar interest. The interest collected through the courses and search histories of the tester and the information on the personality and aptitude tests results of the testers who have similar interest may be previously obtained. Tester information and test question information that are extracted from the personality and aptitude question and interest collection database 320 may be input to a generative language model 330. In this case, the tester information may include a course history, a search history, a list of interests, etc. of the tester, for example. The test question information includes a test question. The test question may include text having a sentence form, such as “generate a question for measuring openness” or “generate a question capable of determining openness and extroversion”, and/or text having a word form, such as “openness”, “extroversion”, “openness and extroversion”, for example. The generative language model 330 may be implemented as GPT-3, BLOOM, or OPT, for example. The generative language model 330 may output psychological test question information including a psychological test question. The psychological test question may include text, for example, “I like to stay at home” or “I enjoy ordering delivery food at home”. The psychological test question information that is output by the generative language model 330 may be provided to a tester terminal 310_2 as a customized question. The tester terminal 310_1 and the tester terminal 310_2 may be the same.

Referring to FIG. 10, a tester terminal 410 may provide a request for a personality and aptitude question to each of a personality and aptitude question and interest collection database 420 and a generative language model 430. The personality and aptitude question and interest collection database 420 may store interest that is collected through courses and search histories of the tester and information on personality and aptitude tests results of testers who have similar interest. The interest collected through the courses and search histories of the tester and the information on the personality and aptitude tests results of the testers who have similar interest may be previously obtained. The personality and aptitude question and interest collection database 420 may return initial test information including an initial test to the tester terminal 410. Tester information and test question information that are extracted from the personality and aptitude question and interest collection database 420 may be input to the generative language model 430. In this case, the generative language model 330 may provide a customized question to the tester terminal 410.

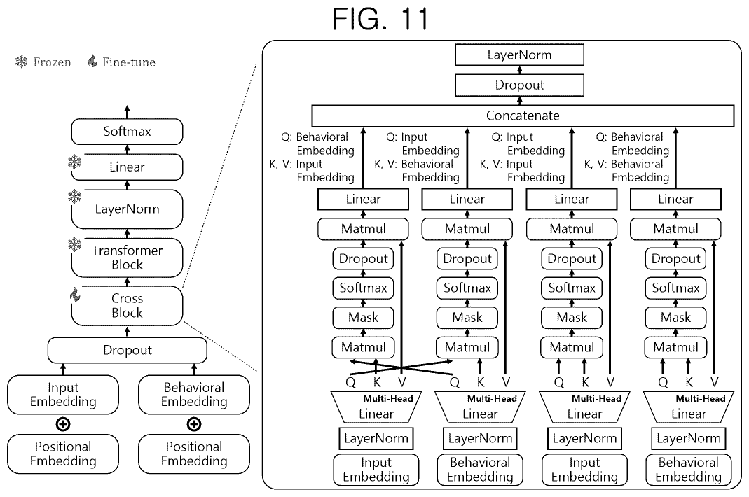

FIG. 11 is a diagram for describing a model architecture having a Decode-Only structure according to the present disclosure.

Referring to FIG. 11, the Decode-Only structure of the present disclosure may be denoted as a GPT structure. A generative artificial intelligence model may perform learning in a fine-tuning way of GPT-2 because the generative artificial intelligence model may include generative pre-training (GPT)-2. The generative artificial intelligence model may include Dropout, a Cross Block, a Transformer Block, LayerNorm, Linear, and Softmax.

The generative artificial intelligence mode may receive input embedding including a description of a question to be generated and behavioral embedding including behavior characteristics of a tester in parallel right before Dropout. That is, two pieces of text may be provided as an input to the generative artificial intelligence model.

The Cross Block may produce pieces of information of the input embedding and the behavioral embedding into one embedding. An operation in which two inputs are considered together may occur in the Cross Block. Specifically, the Cross Block may operate the input embedding and the behavioral embedding by dividing hyper parameters (e.g., H) that are used to calculate a training loss in multi-head attention into two by N when the number of hyper parameters is 2N (N is a natural number). For example, when the number of Hs is 64 in the multi-head attention, the Cross Block may operate the Hs by dividing the Hs into two (32 and 32).

Four embedding processing blocks of the Cross Block may form one pair. Four embedding processing blocks of the Cross Block in which a cross-attention structure and/or a self-attention structure are mixed may form one pair.

In some embodiments, the Cross Block may include a first embedding processing block, a second embedding processing block, a third embedding processing block, and a fourth embedding processing block that form one pair in order to process embedding and that each process each of the input embedding and the behavioral embedding as a query, a key, and a value.

For example, the first embedding processing block may be implemented as an attention structure that processes behavioral embedding of the second embedding processing block as a query and that processes its own input embedding as a key and a value. The second embedding processing block may be implemented as an attention structure that processes the input embedding of the first embedding processing block as a query and processes its own behavioral embedding as a key and a value. The third embedding processing block may be implemented as a self-attention structure that processes its own input embedding as a query, a key, and a value. The fourth embedding processing block may be implemented as a self-attention structure that processes its own behavioral embedding as a query, a key, and a value.

The input embedding and the behavioral embedding having different Q vectors, K vectors, and V vectors may be subjected to a cross-attention operation, concatenated and input as one.

When a user input is fed back, “frozen” may remain without any change, and only “fine-tune” part (e.g., the Cross Block) may be operated. Accordingly, real-time can be achieved.

The existing only decoder model, for example, a GPT-3 structure may be subsequently applied to the present disclosure.

Finally, a question according to the input may be generated by a generative language model.

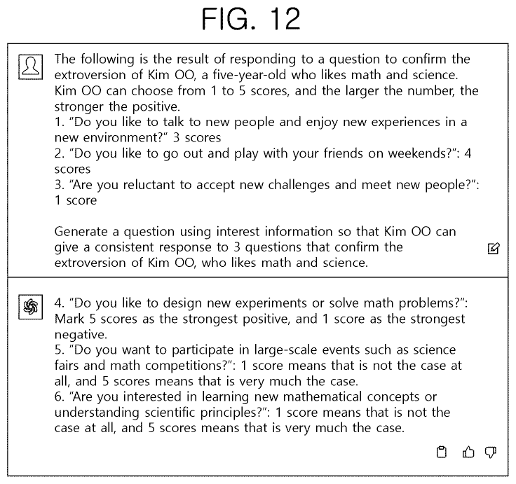

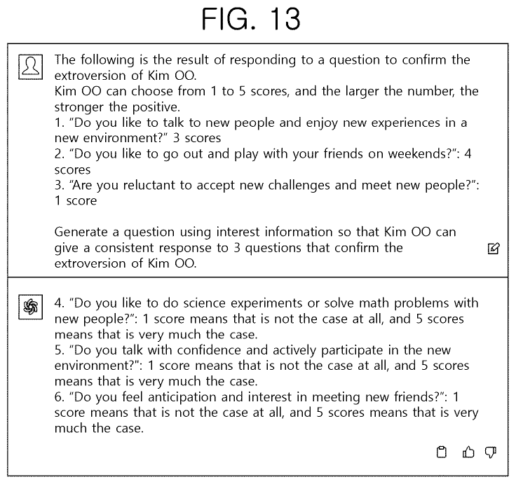

FIGS. 12 and 13 are diagrams for describing embodiments in which a question is generated by using a generative artificial intelligence model of the present disclosure.

Specifically, an embodiment of FIG. 12 is an embodiment in which an actual question is generated when information of a user is inserted. An embodiment of FIG. 13 is an embodiment in which an actual question is generated when information of a user is not inserted. In order for outputs according to the embodiments of FIGS. 12 and 13 to be illustratively checked, a prompt tuning method may be used, but the present disclosure is not limited thereto. Learning may be performed in a fine-tuning way.

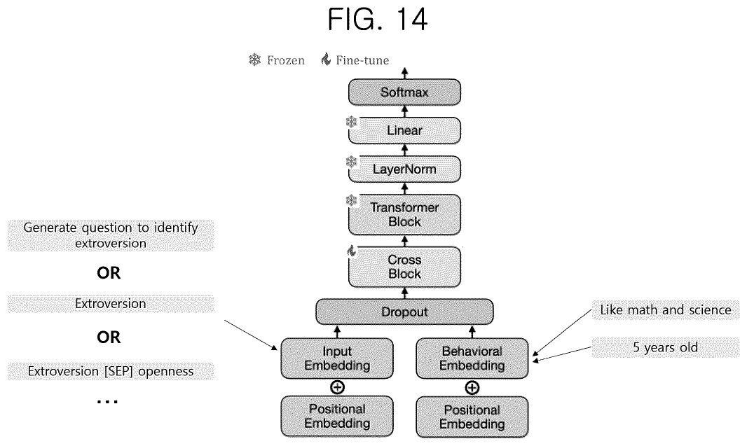

FIG. 14 is a diagram for describing input values for input embedding and behavioral embedding according to the present disclosure.

An embodiment of FIG. 14 illustrates a case in which a user's interest has been inserted into a prompt.

Interest information illustrated in FIG. 14 may be inserted into a generative language model in the form of behavioral embedding. Such interest information may be used for the training and inference of the generative language model. A factor (e.g., “extroversion”) to be known does not need to be essentially one, and a question capable of searching for N factors at once may be generated.

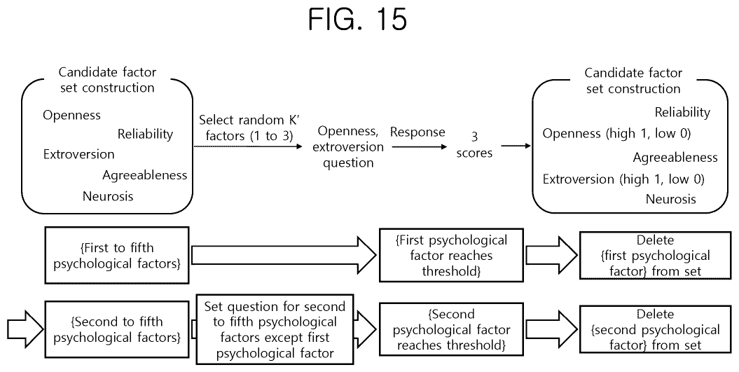

FIGS. 15, 16, 17, and 18 are diagrams for illustratively describing end conditions according to the present disclosure.

Referring to FIGS. 15 to 18, the generative artificial intelligence model may delete a specific factor from a candidate factor set construction including a plurality of candidate factors if a tester responds to a question corresponding to the specific factor in the candidate factor set construction by a predetermined number or more.

Referring to FIGS. 15 to 18, for example, the candidate factor set construction may include various candidate factors, such as “openness”, “reliability”, “extroversion”, “agreeableness”, and “neurosis”.

The generative artificial intelligence model may delete a specific factor from the candidate factor set if the tester responds to more than K questions with respect to the specific factor in the same direction. Referring to FIG. 15, for example, when first to fifth psychological factors are selected and the first psychological factor reaches a threshold, the first psychological factor may be deleted from the set. After that, when a question for the second to fifth psychological factors except the first psychological factor is provided to the tester and the second psychological factor reaches a threshold, the second psychological factor may be deleted from the set.

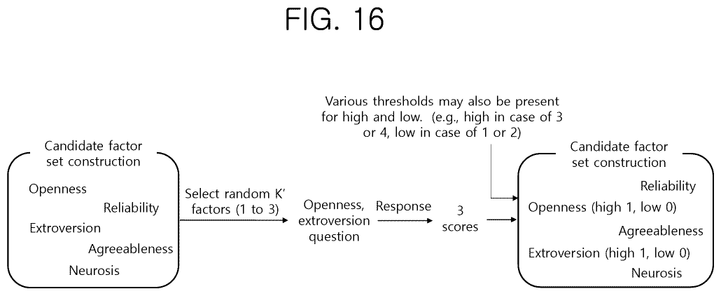

Referring to FIG. 15, for example, factors, for example, “openness” and “extroversion” may be randomly selected in a candidate factor set construction. Accordingly, a response to “openness” and “extroversion” may be generated. A score, for example, 3 points may be assigned to the response. In the candidate factor set construction, a score “high 1” may be assigned to “openness” and “extroversion”. Referring to FIG. 16, for example, various thresholds may be present for “high” and “low”. For example, it may be high in case of 3 or 4 points, and it may be low in case of 1 or 2 points, etc.

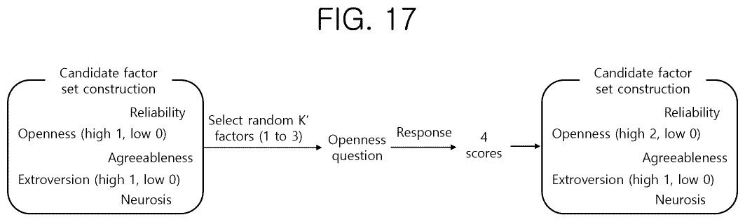

Referring to FIG. 17, for example, in a candidate factor set construction, “openness” may be selected, and a question for “openness” may be generated. A corresponding response may be generated, and a score, for example, 4 points may be assigned to the response. In this case, a score “high 2” may be assigned to “openness”.

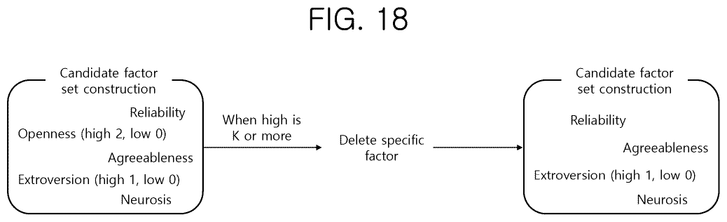

The generative artificial intelligence model may repeat an operation of deleting a factor until all candidate factors in a candidate factor set construction are deleted. Until a set of candidate factors disappears, the factors may be deleted from the set one by one when the score of the factor reaches a threshold. That is, when the number of highs of a factor is K or more in a candidate set construction, a corresponding factor may be deleted. Referring to FIG. 18, for example, “openness” may be deleted from the candidate set construction.

Accordingly, understanding an object of a question may become difficult by putting various factors into one question. For example, if a question of “I like to stay at home” is given to a tester, the tester may easily understand that the object of the question is “openness”, such that 3 or 4 (yes or very much so) may be intentionally selected by the tester. However, if a question of “like to eat at home, but I feel happier when I eat with my friend.” is given to a tester, the tester may not clearly understand that the object of the question is “openness” and “introversion”, such that the intentional selection of a score may be prevented.

As described above, it is possible to reduce the number of questions for deriving the results of a psychological test when a question is provided in real time. That is, there are effects in that the time and costs can be efficiently reduced.

Meanwhile, the disclosed embodiments may be implemented in the form of a recording medium in which a computer-executable instruction is stored. The instruction may be stored in the form of a program code, and may perform an operation of the disclosed embodiments by generating a program module when the instruction is executed by the processor. The recording medium may be implemented as a computer-readable recording medium.

The computer-readable recording medium includes all types of recording media in which a computer-interpretable instruction has been stored. For example, the computer-readable recording medium may include read only memory (ROM), random access memory (RAM), a magnetic tape, a magnetic disk, flash memory, an optical data storage device, etc.

### INDUSTRIAL APPLICABILITY

The present disclosure may be usefully used in practicing a customized test for each user by generating a question in which the characteristics of a person have been incorporated by using artificial intelligence.

Although the preferred embodiments of the present disclosure have been described above, those skilled in the art in the art will appreciate that the present disclosure may be modified and changed in various ways without departing from the spirit and scope of the present disclosure written in the claims described later.

1. A server comprising:
a communication unit configured to communicate with a terminal;
a database configured to store tester information;
a memory configured to store artificial intelligence model data for a generative artificial intelligence model; and
a processor configured to generate personality and aptitude question information suitable for characteristics of a person from personal behavior characteristic information and existing question information by using the generative artificial intelligence model,
wherein the processor
receives tester question information and a request for a personality and aptitude question for personality and aptitude tests from the terminal,
inputs, to the generative artificial intelligence model, the tester information corresponding to the terminal, which are extracted from the database, and the tester question information, and
transmits, to the terminal, the personality and aptitude question information suitable for the characteristics of a tester who uses the terminal, which is provided as an output of the generative artificial intelligence model, through the communication unit.

2. The server of claim 1, wherein the database previously stores the tester information comprising at least one of a course history, search history, and list of interests of the tester.

3. The server of claim 2, wherein the generative artificial intelligence model comprises Dropout, a Cross Block, a Transformer Block, LayerNorm, Linear, and Softmax, and receives input embedding comprising a description of a question to be generated and behavioral embedding comprising the behavior characteristic of the tester in parallel right before the Dropout.

4. The server of claim 3, wherein the Cross Block produces pieces of information of the input embedding and the behavioral embedding into one embedding.

5. The server of claim 4, wherein the Cross Block comprises a first embedding processing block, a second embedding processing block, a third embedding processing block, and a fourth embedding processing block that form one pair in order to process embedding and that each process each of the input embedding and the behavioral embedding as a query, a key, and a value.

6. The server of claim 5, wherein:
the first embedding processing block is implemented to have an attention structure in which behavioral embedding of the second embedding processing block is processed as a query and its own input embedding is processed as a key and a value,
the second embedding processing block is implemented to have an attention structure in which the input embedding of the first embedding processing block is processed as a query and its own behavioral embedding is processed as a key and a value,
the third embedding processing block is implemented to have a self-attention structure in which its own input embedding is processed as a query, a key, and a value, and
the fourth embedding processing block is implemented to have a self-attention structure in which its own behavioral embedding is processed as a query, a key, and a value.

7. The server of claim 6, wherein the generative artificial intelligence model deletes a specific factor from a candidate factor set construction comprising a plurality of candidate factors if the tester responds to a question corresponding to the specific factor in the candidate factor set construction by a predetermined number or more.

8. The server of claim 7, wherein the generative artificial intelligence model repeats an operation of deleting a factor until all the candidate factors included in the candidate factor set construction are deleted.

9. A method of generating personality and aptitude question information suitable for characteristics of a person from personal behavior characteristic information and existing question information by using a generative artificial intelligence model, the method comprising:
receiving tester question information and a request for a personality and aptitude question for personality and aptitude tests from a terminal;
inputting, to the generative artificial intelligence model, tester information corresponding to the terminal, which are extracted from a database, and the tester question information; and
generating personality and aptitude question information suitable for characteristics of a tester who uses the terminal, which is provided as an output of the generative artificial intelligence model.
